(19) United States
(12) Patent Application Publication
(10) Pub. No.: US 2005/0021142 A1
(43) Pub. Date: Jan. 27, 2005

(54) Method of manufacture, installation, and system for a sinus lift bone graft

(75) Inventors: Scott D. Ganz, River Vale, NJ (US); Peter A. Materna, Metuchen, NJ (US); Thomas J. Bradbury, Yardley, PA (US); Roger C. Stikeleather, Doylestown, PA (US); Adolphe H. Youssef, Kendall Park, NJ (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Therics, Inc., Princeton, NJ (US)

(21) Appl. No.: 10/789,367

(22) Filed: Feb. 26, 2004

Related U.S. Application Data
(60) Provisional application No. 60/450,410, filed on Feb. 26, 2003.

(51) Int. Cl. A61B 17/58

(52) U.S. Cl. 623/16.11

(57) ABSTRACT

A bone graft that is made at least partially of synthetic material or demineralized bone matrix and is of a suitable shape to fill a lower portion of a patient's maxillary sinus. The graft may be manufactured to patient-unique dimensions that may be determined radiographically prior to surgery and prior to manufacturing of the bone graft. Other aspects of the invention are a method of manufacture of the bone graft, and methods of installing the bone graft. The installation may make use of patient-unique templates to guide certain cutting operations. Another aspect of the invention is a kit comprising the bone graft, tools for its installation, templates and possibly other surgical items.

### BACKGROUND OF THE INVENTION

1. Field of the Invention

The present invention relates to sinus lift bone graft, and more particularly, method of manufacturing the bone graft, a method of installing the bone graft and a system for implementing the same.

2. Description of the Related Art

An endosseous implant (EDI) comprises an implant base that is installed directly into the bone of a patient's mandible or maxilla, and an abutment post that attaches to the implant base, and a tooth prosthesis that attaches to the abutment post. Implant dentistry has become a practical restorative method with a high reliability and success rate. For an EDI to be successful it is necessary that the implant base have an appropriate length that is supported by intimate contact with bone. For implant bases which are installed in the maxilla, this thickness of available bone is defined in part by the floor of the maxillary sinus. In some cases, such as due to bone resorption following tooth loss, the thickness of the maxilla is inadequate to support an implant base. FIG. 2 is a three-dimensional view looking inside a maxillary sinus from above, showing a maxilla which is fairly thin and has not received a sinus lift and which has implant bases 210, 220 installed in an excessively thin region such that the implant base protrudes into the sinus cavity and is inadequately supported. FIG. 3 is a cross-sectional view along lines 3-3 of FIG. 2 illustrating the protrusion of the implant base 220 into the sinus cavity 310. This situation could lead to mechanical loosening and failure of the implant.

In situations such as these, it has still been possible to successfully install an implant base if the maxilla could be thickened by adding bony material above the maxilla onto the floor of the maxillary sinus cavity, a procedure called sinus elevation or sinus lift. Existing sinus lift procedures are described in “An Illustrated Guide to Understanding Dental Implants,” by Scott D. Ganz, D.M.D. (1993).

Additions of bony material is illustrated in FIG. 4 through 6. As a starting point of the surgical procedure, FIG. 4 illustrates that an access window 410 has been created by cutting through the buccal cortical plate, which separates the maxillary sinus from the buccal cavity. Similarly, FIG. 5 illustrates that a window or opening has been cut into the buccal cortical plate, and is about to be infractured into the sinus. The window can be either excised or infractured. FIG. 6 shows that the window has been infractured and also the membrane which lines the interior of the maxillary sinus has been elevated from the walls and floor of the sinus.

Depending on the location of the intended bone graft for a particular patient, a septum of the maxillary sinus may be removed to make room for the filler that is to be installed, or to provide a path along which the filler can be brought into its desired position. Alternatively, miscellaneous tissue inside the maxillary sinus, such as soft tissue, is removed from the maxillary sinus.

Next, in some procedures, an appropriate lower portion of the sinus cavity has been filled with a formable filler material that is intended to become bone or result in the formation of natural bone. For example, the formable material has sometimes been a paste or putty comprising demineralized bone matrix, bone chips, other components derived from bone, etc. However, such formable material has not always remained where it has been placed and has not always integrated sufficiently well with the existing bone to form a strong foundation for installation of an implant base. Sometimes the formable material has stayed in place and has successfully integrated with existing bone but has later resorbed. This type of procedure has required a time for healing and osseointegration after placement of the graft, before installation of the implant base. With formable material, the installation of the implant base has had to be performed in a later surgery.

In other sinus lift procedures, after the same preparatory steps, an appropriate lower portion of the sinus cavity has been filled by a rigid graft material of natural origin such as allograft or autograft bone. Because the autograft or allograft has been a solid material, such a procedure has avoided the migration problem experienced with formable material. However, the bone installed in such a procedure has still been subject to possible resorption. As is usually the case with such sources of bone material, the use of allograft bone has introduced the possibility of disease transmission from the donor, and the use of autograft bone has involved the extra inconvenience, pain and expense of the surgery at a second site in the same patient for harvesting of bone. Also, the use of such sources of bone material has involved time-consuming carving and shaping of the bone graft during the surgery.

In this type of procedure, the decision as to whether the implant base can be installed during the sinus lift surgical procedure has depended on what thickness of bone was present in the maxilla at the time of surgery. All of the above-mentioned procedures have still involved the possibility of resorption of implanted bone material, which would represent a re-occurrence of the original problem.

In regard to surgical technique for use with grafts of any rigid material, both preparation of the sinus cavity and shaping of the bone graft have typically been performed using localized cutting tools such as small burrs. Typically, sequential on the spot cutting and fitting have been performed so that the graft fits with the appropriate portion of the sinus cavity. Typically the graft has had to be inserted, removed, adjusted and re-inserted a number of times during a surgical procedure, with decisions being made as the surgery progressed.

Accordingly, there remain multiple needs for improvement in procedures for sinus lift and installation of implant bases into the maxilla. It would be desirable to avoid the problems of migration of non-solid material. It would be desirable to avoid resorption of any type of implanted material. It would be desirable to avoid the problems of second site surgery or possible disease transmission that are inherent with autograft and allograft, respectively. It would be desirable to make the surgical process as efficient as possible by reducing the amount of unrehearsed cutting and fitting which has to take place during surgery. It would be desirable to improve the fit between bone graft from any source and the sinus cavity into which the bone graft is placed, so as to promote integration of natural bone with the bone graft. It would be desirable to minimize the number of surgical procedures that a patient must undergo.

### SUMMARY OF THE INVENTION

An aspect of the invention is a bone graft which is made at least partially of synthetic material or demineralized bone matrix and which may be of a suitable shape, and which may be manufactured in the suitable shape, to fill a lower portion of a patient's maxillary sinus. The graft may be manufactured to patient-unique dimensions that may be determined radiographically prior to surgery and prior to manufacturing of the bone graft. Other aspects of the invention are a method of manufacture of the bone graft, and methods of installing the bone graft. The installation may make use of patient-unique templates to guide certain cutting operations. Another aspect of the invention is a kit comprising the bone graft, tools for its installation, templates and possibly other surgical items.

### BRIEF DESCRIPTION OF THE FIGURES

FIG. 1 illustrates the three-dimensional printing process in accordance with the prior art.

FIG. 2 shows a three-dimensional image of bone in a patient in which two implant bases protrude into the maxillary sinus cavity.

FIG. 3 is a schematic cross section of FIG. 2 along lines 3-3.

FIG. 4 shows the first step of a sinus lift surgical procedure, in which a window into the maxillary sinus may be created.

FIG. 5 is an image of a next step of a sinus lift surgical procedure, in which the window of FIG. 4 is about to be folded inward.

FIG. 6 is an image of a next step of a sinus lift surgical procedure, in which the window has been folded inward and the adjacent tissue has been lifted up.

FIG. 7 is an image of another step of a sinus lift surgical procedure, in which the bone graft is in place in the maxillary sinus and two implant bases are installed into it in accordance with principles of the present invention.

FIG. 8 is a view of a portion of a patient's maxilla and sinus region made out of plastic as a surgical model, with a bone graft of the present invention placed in the sinus cavity in accordance with principles of the present invention.

### DETAILED DESCRIPTION OF THE INVENTION

Bone Graft

An aspect of the present invention is the bone graft itself. As used herein, the term bone graft is intended to include natural bone (from any source), and processed components of natural bone, and synthetic material of all kinds, and combinations thereof, in a form that is rigid. Some specific types of bone graft are an aspect of the present invention. The bone graft of the present invention may be described both by its geometry and by its material composition.

The bone graft of the present invention may be made of a rigid material, so that it can have and retain definite shape and dimensions, as opposed to being formable. One possibility is that the bone graft may be manufactured in a non-specific shape intended to be shaped during surgery by removing material from it. Another possibility is that the bone graft may be manufactured to approximate dimensions but may be modified during surgery by removing material from it in local places for dimensional adjustment as required for good fit. Another possibility is that the bone graft may be manufactured to patient-unique dimensions in advance of surgery so exactly that no adjustment or removal of material from it need be made during-surgery.

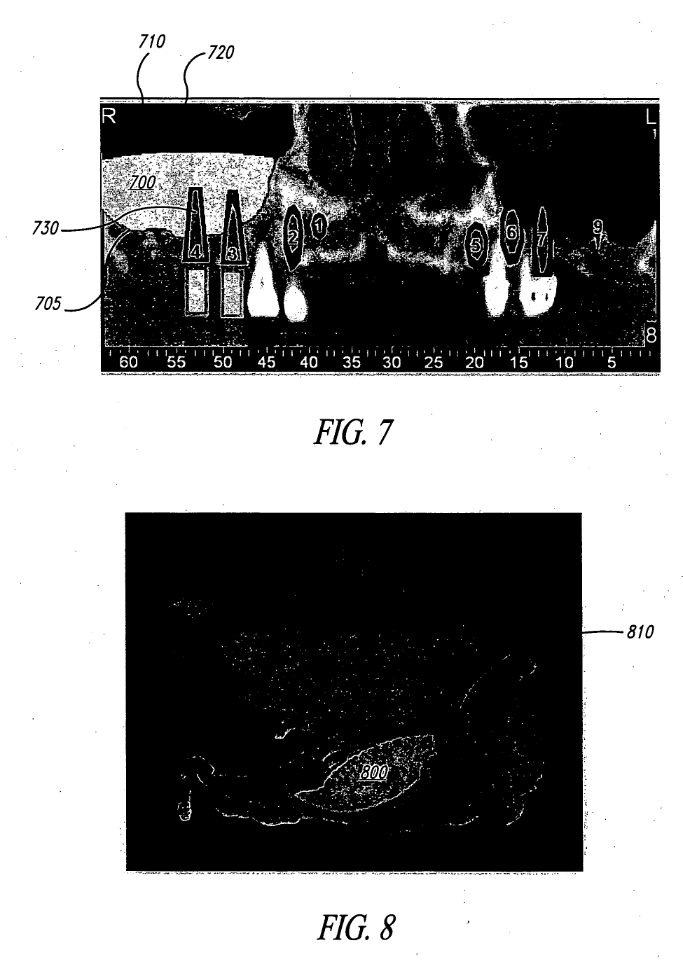

As illustrated in FIG. 7, a bone graft 700 for a sinus lift may have a surface which when installed is adjacent to natural bone, which appears in the illustrations as a lower surface 705, and a surface which when installed is not adjacent to natural bone, which appears in the illustrations as the upper surface 710. The upper surface 710 may be flat or of almost any shape. The lower surface 705 of the bone graft 700 may be shaped so as to have a defined spatial relationship with the shape of the corresponding portion of sinus cavity 720. The sinus cavity 720 to which the bone graft 700 has a spatial relationship either may be the natural shape of the sinus cavity or may be the natural shape of the sinus cavity as it is anticipated it would be modified by removal of a septum or soft tissue or other structure.

The defined spatial relationship may mean that the bone graft may be closely fitting to the appropriate portion of the sinus cavity, to within a close tolerance. Alternatively, it may mean that the bone graft could have a predetermined gap, which may everywhere be maintained to within a close tolerance, with respect to the corresponding portion of the sinus, or the bone graft could have a predetermined amount of interference, which may everywhere be maintained to within a close tolerance, with respect to the corresponding portion of the sinus. With the bone graft manufacturing method and the surgical methods described herein, it is believed that a tolerance of better than 0.4 mm may be achieved on the relative dimensions of the bone graft and the corresponding portion of the sinus cavity. This tolerance may be applied in the form of either gap or interference as desired, or even a combination of gap in some places and interference in other places.

The bone graft may comprise additional geometric features other than its external shape. The bone graft may comprise one or more features such as holes to assist in the installation of one or more implant bases. The position and orientation of those features may be unique to an individual patient. The bone graft may be manufactured with one or more holes already in it for use by a surgical screw or similar attachment device. The bone graft also may include features that are conducive to gripping of the bone graft as it is carried to or installed in the sinus cavity.

It is possible that the bone graft may be created in more than one piece that cooperate with each other to make up the desired shape of bone graft. For example, the bone graft may be manufactured in pieces such that moving or fitting the bone graft into its intended position may be easier, or may require a smaller window, than if the bone graft were manufactured as a single piece. Manufacture of a multi-piece bone graft using methods of the present invention (as described elsewhere herein) is essentially just as easy as manufacture of a single-piece bone graft, as long as appropriate software instructions for 3DP can be generated.

At a smaller dimensional scale, the bone graft may comprise any of various sorts of channels or similar geometric features which may be conducive to osseintegration. The bone graft may comprise channels within its interior. The bone graft may comprise channels or patterns on at least some exterior surfaces, such as surfaces of the bone graft that are intended to face natural bone; the bone graft may further include a second pattern or surface such as for soft tissue. The bone graft may comprise composition that is different at a surface that is intended to face natural bone, as compared to elsewhere in the bone graft. If it is desired that the bone graft have a geometry or composition at its surface which is different from its geometry or composition interiorly of the surface, then the combination of various aspects of the present invention, including the ability to custom-manufacture a bone graft with prescribed detail, tailored to dimensions within the patient's sinus, provides confidence that there will not be a need to remove material from, and thereby disturb the designed surface features of, the bone graft at the time of installation in the patient.

The bone graft may comprise a matrix material that exists in the form of particles joined to each other so as to form a three-dimensionally interconnected network. In terms of material composition, the matrix material may be or may include a synthetic material. The matrix may be made of a ceramic material that may resemble materials found in natural bone and in particular may be a compound comprising calcium and phosphorus. If the bone graft is made entirely of synthetic material, that would avoid the possibilities of disease transfer associated with the use of donor bone (allograft) and would avoid the second site surgery associated with autograft.

The matrix material may be nonresorbable. Such a bone graft may be made of or may include nonresorbable hydroxyapatite. The property of nonresorbability may be useful for combating a situation in which natural bone has resorbed. A nonresorbable material that is porous may tend to remain permanently in place while still allowing or encouraging natural bone to grow into its void spaces, thereby resulting in a combination of at least some of the strength of natural bone together with a tendency not to resorb.

Alternatively, the matrix material may be resorbable or have a resorbable component. In this situation, the material may be or may include tricalcium phosphate. It is possible that both nonresorbable and resorbable materials may be used in the bone graft. The matrix may contain both hydroxyapatite and tricalcium phosphate, and the proportions of those two substances may vary from one place to another. The matrix material may be ceramic, as just described. Alternatively, it is also possible that the matrix material may be or may comprise demineralized bone matrix (DBM), with particles of DBM being joined by a binder substance. The bone graft may comprise polymer particles as the matrix material.

Because the matrix may be porous, it may have pores that may be three-dimensionally interconnected. The porosity and the pore size or pore size distribution may be chosen so as to encourage natural bone to grow into the bone graft. The matrix of the bone graft may have pores whose size may be described as a distribution of pore volume as a function of pore size that has a mode at a pore dimension of between 10 micrometers and 25 micrometers. The porosity of the bone graft, which is the fraction of space not occupied by the matrix, may be in the range of from 20% void to 60% void.

The bone graft may further include at least one other material occupying at least some of the pores of the matrix. The bone graft may be osteoconductive or osteoinductive and may comprise additives to give it properties of osteoconduction or osteoinduction, for example, additives which occupy at least some of the pores of the matrix. The bone graft may include demineralized bone matrix (DBM) occupying some of the pores of the matrix. Other possible additive materials can include the patient's own blood products, and any of a number of possible growth-stimulating or biological additives, as described in the patent application referenced below.

The pores in the matrix of the bone graft may be partially or fully occupied by a polymer, which may be either resorbable or nonresorbable. An example of a resorbable polymer is poly lactic co-glycolic acid (PLGA), and an example of a non-resorbable polymer is poly methyl methacrylate (PMMA). Other examples of each type are given in the patent application referenced below. The polymer may be or may include a comb polymer, as described in U.S. Pat. No. 6,150,459 and elsewhere. The presence of material occupying space in the pores of the matrix may be uniform throughout the bone graft or may be concentrated unequally in certain regions of the bone graft.

The bone graft may be capable of being cut, during surgery or around the time of surgery, to dimensions other than the originally manufactured dimensions, using either powered cutting tools or hand-held cutting tools. The ability to cut or carve the bone graft may be useful for dimensional adjustment and improving fit between the bone graft and the sinus cavity during surgery, if that becomes necessary.

With regard to its material composition, its design and any other aspects, the bone graft may include any of the features, properties and the like, which are described in U.S. patent application Ser. No. 60/286,564; hereby incorporated by reference.

FIG. 8 shows a plastic model of the maxilla 810 and nearby portion of a patient's skull, sectioned at a horizontal plane. The model has been created by stereolithography from a three dimensional solid model originally derived from radiological data. Also shown, in white, is a bone graft 800 of the present invention which was made by the manufacturing method of the present invention and which fits closely to the model of the maxilla. This illustrates the close fit that is possible between the existing bone and the bone graft of the present invention, as manufactured and without repetitive adjustment of dimensions.

Method of Installation of Bone Graft

Another aspect of the present invention is a method of performing sinus lift. The method may comprise installing the bone graft of the present invention.

In preparation for the surgical procedure, the patient may be radiographed and dimensions may be determined of the sinus cavity and maxilla of the patient. The information may be mathematically represented as a three-dimensional solid model. Using that dimensional information, the bone graft may be designed to patient-unique dimensions. The bone graft may be designed fit a portion of the bone structure that was measured in the radiograph. The bone graft may then be manufactured as described elsewhere herein.

During the surgical procedure of the present invention, the window in the maxillary sinus may be cut by determining its location and/or size and/or shape using a template that may be unique to a particular patient, with its dimensions being decided as a result of radiographic data from that particular patient. The template may define its own overall position relative to particular teeth or other features in the mouth of the patient.

Following this, as is conventionally done, the window may be infractured or may simply be excised. Cutting may be done with bone-cutting tools. If it is necessary to prepare the interior of the sinus cavity, such as by removing soft tissue or removing a septum, that removal can be performed as is conventionally done. For removal of soft tissue, it is possible to use none-bone-cutting tools, either hand-held or powered, which are capable of cutting soft tissue but are not sufficiently hard to cut bone.

After the sinus cavity in the maxillary sinus has been prepared as just described, the bone graft of the present invention may be moved into the sinus cavity. The fit between the bone graft and the prepared sinus cavity may be adequate such that no adjustment is needed. Alternatively, it is possible that some adjustment to improve fit may have to be done during the surgical procedure, such as by removing material from the bone graft in selected locations or removing material from the sinus cavity. It may be necessary to repeatedly bring the bone graft into the sinus cavity, check fit, remove the bone graft and adjust either the bone graft or the sinus cavity.

The geometry of the sinus cavity and the design of the bone graft may be such that after the bone graft has been brought into the sinus cavity, the bone graft may be maintained in sufficient contact with adjacent natural bone simply by virtue of its shape and dimensions. Alternatively, it is possible that the bone graft may require some anchoring in order to maintain it in contact with the adjacent natural bone. If such anchoring is needed, appropriate anchoring may be performed at this point during the surgical procedure, such as installation of surgical screws, and appropriate features may be provided in the design of the bone graft to accommodate such anchoring.

An implant base for an endosseous implant may be installed in both the maxilla and the bone graft during the same surgical procedure in which the bone graft itself is installed in the patient. In this event, the implant base may also serve as anchoring to keep the bone graft in place. Preparation of the implant base site may be performed using a template that may be patient-unique. However, it is not required that the implant base be installed during the same surgical procedure as the bone graft. Alternatively, installation of the implant base may be done separately in a later procedure after there has been some amount of healing and integration of the bone graft with natural bone.

During the later stages of the surgical procedure, appropriate surgical substances may be applied as appropriate. Antiseptic and/or antibiotic may be applied before the bone graft is put into place permanently. Even though the bone graft may be a close fit with the sinus cavity, it is possible that formable material such as putty containing bone-derived substances may be applied for filling possible gaps between the bone graft and the adjacent bone or simply to improve the interaction between the bone graft and the adjacent bone. A surgical membrane such as Gore-Tex or collagen may be used to inhibit the growth of soft tissue in certain places. The window may then be closed with bone from the buccal cortical plate of the maxilla, or might just be covered with gingival tissue. Suturing may be performed.

If installation of the implant base is performed during the sinus lift procedure, it is also possible that a temporary abutment post or even an abutment post plus a tooth prosthesis may be installed onto the implant base during the same surgical procedure.

FIG. 7 is an X-ray of a patient's maxilla modified to illustrate an implant base 730 together with a bone graft 700 of the present invention, both of them in approximately the position they would occupy as a result of the procedure described herein. Use of templates Other aspects of the method of the present invention are the use of templates. The access for installation of the bone graft may be planned in advance of surgery and, if desired, a window template may be made to guide the creation of the window in the buccal cortical plate. The window template may define the position, shape and dimensions of the window. The window template may be derived at. least in part from patient-unique data, which may be radiographic data. The window template may be coordinated with the design of the bone graft so that the bone graft may fit sufficiently easily through the window described by the template. The window template may take its overall location from one or more teeth or other features in the patient's mouth. The window template may, for example, be made by stereolithography using the same set of solid modeling data used for other aspects of surgical planning. In a procedure of the present invention which uses a patient-unique window template for cutting the window, it is possible to install a bone graft, but it is also possible that infilling of the sinus may be performed entirely with formable material.

If it is planned to install implant base(s) during the same surgical procedure as the sinus lift, the installation of the implant base(s) may be aided by an implant base template suitable to guide drills or other tools for installation of an implant base. The implant base template may be derived at least in part from patient-unique data that may be radiographic data. The implant base template may take its overall location from one or more teeth or other features in the patient's mouth.. The implant base template may, for example, be made by stereolithography using the same set of solid modeling data used for other aspects of surgical planning, and may comprise drill bushings to locate and orient drills. If more than one drill is used in succession to prepare the site for the implant base, more than one implant base template may be created and used.

Method of Manufacture of Bone graft

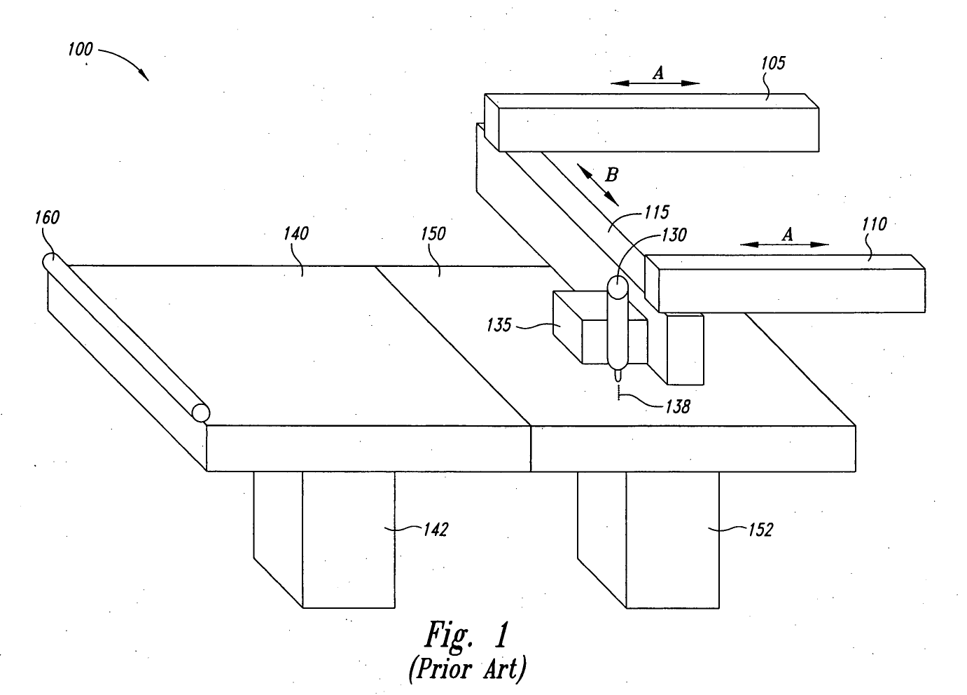

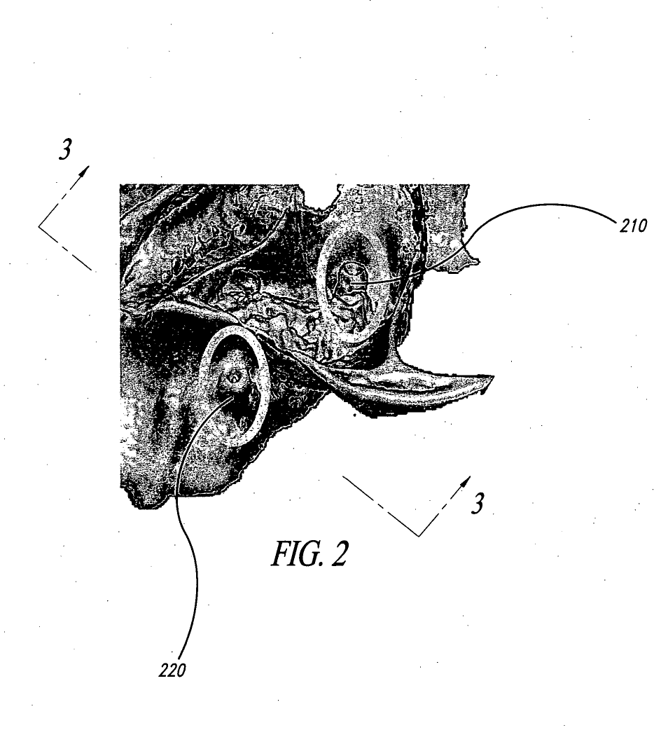

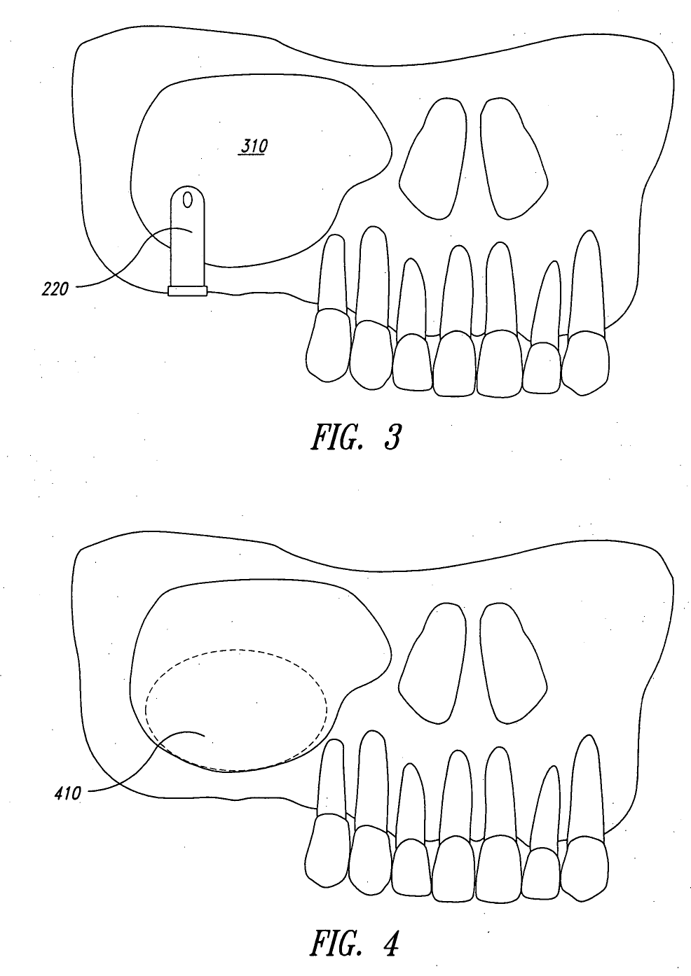

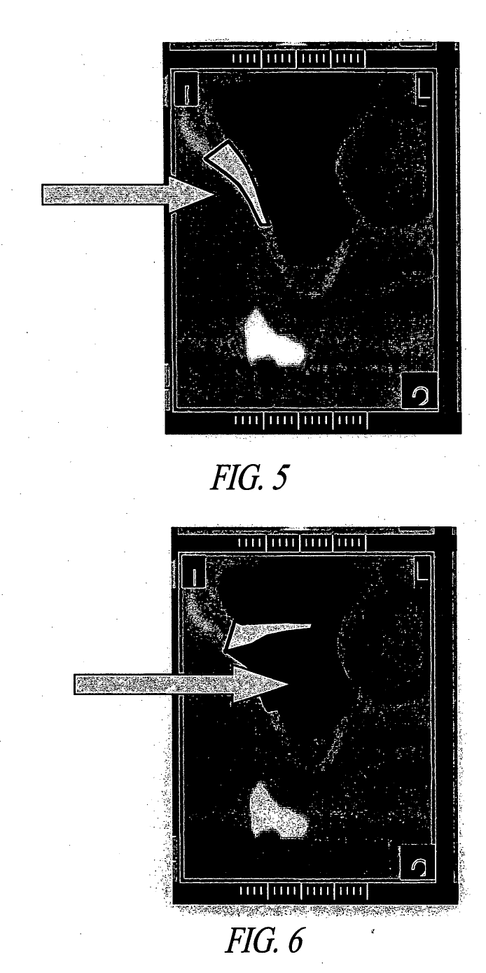

The bone graft of the present invention may be manufactured by methods that include three-dimensional printing (3DP). Three-dimensional printing described in U.S. Pat. No. 5,204,055 and elsewhere, is the manufacture of objects by assembling them from powder in a layer-by-layer fashion. FIG. 1 illustrates an exemplary embodiment of a three-dimensional printing apparatus 100 in accordance with the prior art. The apparatus 100 includes a roller 160 for rolling powder from a feed bed 140 onto a build bed 150. Vertical positioners, 142 and 152 position the feed bed 140 and the build bed 150 respectively. Slow axis rails 105, 110 provide support for a printhead 130 in the direction of slow axis motion A, and fast axis rail 115 provides support for the printhead 130 in the direction of fast axis motion B. The printhead 130 is mounted on support 135, and dispenses liquid binder 138 onto the build bed 150 to form the three-dimensional object.

In selected places powder particles are joined to other powder particles and to other bound regions by the action of a binder liquid that may be dispensed from a dispenser that may resemble an ink-jet printer. Binding can occur as a result of a non-volatile substance being deposited by the binder fluid or dissolved by the binder fluid as the binder fluid lands on the powder bed, or can occur as a result of dissolution of powder particles followed by re-solidification. Unbound powder supports bound regions during printing and can later be removed after completion of 3DP. If appropriate geometric description is available and appropriate software instructions for 3DP can be generated, geometrically complicated articles can be made essentially just as easily as geometrically simple articles can be made.

Implantable bone substitutes can be made by. using powder that is a ceramic substance and may resemble substances found in natural bone. Possible substances include hydroxyapatite, tricalcium phosphate and other calcium-phosphorus compounds.

Such articles may involve a sintering step after the completion of 3DP. The sintering may be partial sintering, which may be carried out at a combination of temperature and time such that the powder particles partially join directly to each other and yet leave some porosity between them. During the heating leading up to partial sintering, the binder substance may exit from the article in the form of vapor or gaseous decomposition products. During partial sintering the powder particles themselves may soften so as to partially join each other, while still leaving a controlled amount of porosity between them. If a ceramic-sintering step is used, it is likely to be the highest-temperature step in the entire manufacturing sequence, and to be the step that is incompatible with organic substances. If such substances are desired in a sintered ceramic bone graft, they may be added after completion of a sintering step.

Implantable bone substitutes can also be made of or can contain non-ceramic substances including demineralized bone matrix (DBM) and polymers. It is possible that the bone graft may be made by spreading powder which is or comprises demineralized bone matrix (DBM), i.e., DBM would be the matrix material, and joining those powder particles to each other using a binder substance. Because of the temperature limitations of DBM, the manufacture of such an article would not involve sintering at elevated temperature after 3DP. It is similarly possible that the article could be manufactured by spreading powder particles of polymer and joining them to each other either by dissolution/resolidification or by a binder substance. Again, there would be temperature limitations much lower than the temperatures used in sintering ceramics.

Addition of biological substances, polymers or other temperature-sensitive substances to the bone graft may be performed after the sintering step if a sintering step is used, or after the basic 3DP-manufacturing step. Such addition of biological substances may be performed, for example, by dipping the bone graft into a liquid solution or by infusing liquid into some or all of the bone graft. In the case of polymers, the polymer may be dissolved in a solvent such as chloroform, which may then be allowed to evaporate.

These techniques and others are further described in co-pending commonly assigned U.S. patent application Ser. No. 60/286,564. In regard to designing the bone graft uniquely for a particular patient, such as from radiographic data, appropriate techniques are described in co-pending commonly assigned U.S. patent applications Ser. No. 09/828,504 and Ser. No. 09/972,832. The techniques described therein can also be used for designing templates for use during the surgical procedure, and for manufacturing (if desired) a physical model of an appropriate portion of the patient's skull for surgical planning purposes.

Kit

Another aspect of the present invention is a kit comprising components that may be used during the described surgical procedure.

The kit may comprise at least one bone graft of the present invention intended for implantation in the patient. The dimensions of the bone graft(s) may be coordinated with any or all of: appropriate dimensions of the patient's sinus cavity; the measured extent of bone resorption/degradation in the patient; and dimensions and intended position of an implant base intended to be installed in the maxilla and the bone graft. In addition to a first bone graft intended for implantation into the patient, the kit may further include a duplicate bone graft for use in case of unexpected need during surgery, and/or may include a bone graft which is oversized such that it could be carved or fitted to size during surgery if needed, and/or may even include a bone graft which is a featureless block of material, any of which could be cut to fit during surgery if needed.

The kit may also comprise one or more cutting tools such as for cutting the window. The kit may comprise bone-cutting tools that are suitable for cutting bone. The kit may comprise non-bone-cutting tools, either hand-held or powered, which are suitable for cutting soft tissue but not suitable for cutting bone. The kit may also comprise a window template that guides the cutting of the window by determining the location and/or size and/or shape of the window that is cut in the buccal cortical plate. If the implant base will be installed during the same surgery as the bone graft, the kit may also comprise at least one implant base template for determining the position and direction of drilling for installation of implant bases. For installation of implant bases, the kit may further include the implant base(s), and implant base site preparation tools such as drills, and an implant base installation tool.

The kit may further include a handling tool for placing the bone graft into the sinus. The kit may include a surgical membrane such as GoreTex or collagen suitable to block the growth of soft tissue in desired places. The kit may include surgical screws or similar hardware suitable for attaching the bone graft, and tools suitable for installing the surgical screws. The kit may include antiseptics and/or antibiotics. The kit may further include formable filler materials suitable for filling possible gaps between the bone graft and adjacent bone, or, alternatively, for use as the entire filler material. The kit may include suture materials. The kit may be designed so that it, or appropriate components of it, are sterilized and packaged or otherwise maintained in a sterile condition.

Further Comments

It can be appreciated that the bone graft of the present invention is a synthetic material conducive to the ingrowth of natural bone that has not heretofore been available for use in sinus lifts. The described bone graft is a rigid article which may be made partially or entirely of synthetic material, and is conducive to the ingrowth of natural bone. The bone graft will not migrate. If the bone graft comprises hydroxyapatite, the hydroxyapatite itself does not resorb, meaning that the bone graft will not completely disappear. The bone graft can include an extent of designed detail, as far as geometry and/or composition, which has not heretofore been available. The bone graft can provide a degree of dimensional matching to the patient's sinus cavity, at the time of manufacture of the bone graft, which has not heretofore been available.

It can also be appreciated that the described bone graft and procedure greatly increase the amount of planning and dimensional determination that can be done in advance of surgery, thereby reducing some of the work which normally has to be done during surgery. The bone graft can be manufactured ahead of time to exact patient-unique dimensions. This can potentially improve the quality of fit between the bone graft and the sinus cavity, perhaps approaching the quality of fit of a filler made entirely of formable material, which should be conducive to bone ingrowth. At the same time, the amount of surgical time and labor required for installing such a bone graft would be relatively small, roughly comparable to the surgical time for installing a formable filler material. This would be achieved without much repetitive cutting and fitting during surgery, and so should not require a long surgical procedure.

It can also be appreciated that the simultaneous use of multiple aspects of the present invention provides abilities not heretofore available. The custom dimensioning and custom manufacture of the bone graft may provide the ability to create a desired fit during surgery with little or no unrehearsed cutting-to-fit or adjustment during the surgical procedure. It becomes possible to design and manufacture a bone graft of precise dimension which has a specified geometry and/or composition at those surfaces which are intended to abut the natural bone of the sinus, and some other different specified geometry or composition internally, and to be confident that the prepared sinus cavity will match closely with the pre-manufactured surface of the bone graft and that there will not be a need to remove material from the surface of the bone graft (which might alter the designed surface geometry or composition) for purposes of fitting.

All patents and applications cited above are incorporated by reference in their entirety. Furthermore, Provisional Patent Application Ser. No. 60/450,411 entitled Method and System for Repairing Endosseous Implants, Such as With a Bone Graft Implant, filed Feb. 26, 2003, and Provisional Patent Application Ser. No. 60/450,407 entitled Method of Manufacture, Installation and System for an Alveolar Ridge Augmentation Graft, filed Feb. 26, 2003, are hereby incorporated by reference.

The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. Aspects of the invention can be modified, if necessary, to employ the process, apparatuses and concepts of the various patents and applications described above to provide yet further embodiments of the invention. These and other changes can be made to the invention in light of the above detailed description.

From the foregoing it will be appreciated that, although specific embodiments of the invention have been described herein for purposes of illustration, various modifications may be made without deviating from the spirit and scope of the invention. In general, in the following claims, the terms used should not be construed to limit the invention to the specific embodiments disclosed in the specification and the claims, but should be construed to include all methods, apparatus and articles that operate under the claims. Accordingly, the invention is not limited by the disclosure, but instead the scope of the invention is to be determined entirely by the following claims.

1. A bone graft suitable to augment a sinus, wherein the bone graft comprises synthetic material or demineralized bone matrix in a rigid form.

2. The bone graft of claim 1 wherein the bone graft is manufactured to a desired shape or desired dimensions.

3. The bone graft of claim 2 where the shape or dimensions are chosen based on dimensions of a particular patient.

4. The bone graft of claim 2 wherein the shape or dimensions are chosen based on radiographic data from a particular patient.

5. The bone graft of claim 1 wherein the bone graft has external surfaces facing adjacent natural bone in a patient, which have a defined spatial relationship with respect to the adjacent natural bone to within a tolerance of less than 0.4 mm.

6. The bone graft of claim 5 wherein the defined spatial relationship is a fit to within the tolerance.

7. The bone graft of claim 5 wherein the defined spatial relationship is a gap of a defined dimension, to within the tolerance.

8. The bone graft of claim 5 wherein the defined spatial relationship is an interference of defined dimension, to within the tolerance.

9. The bone graft of claim 5 wherein at some place the defined spatial relationship is a member selected from the group consisting of a gap, and a fit and an interference, and at some other place the defined spatial relationship is a different member selected from the same group.

10. The bone graft of claim 1 wherein the bone graft comprises more than one piece which together provide a desired shape.

11. The bone graft of claim 1 wherein the bone graft comprises at least one hole corresponding to an intended position of an implant base.

12. The bone graft of claim 1 wherein the bone graft comprises at least one hole corresponding to an intended position of an attachment device.

13. The bone graft of claim 1, further comprising at least one feature allowing the bone graft to be held by a gripping tool.

14. The bone graft of claim 1 wherein the bone graft comprises a matrix of particles joined to each other forming a three-dimensionally interconnected network.

15. The bone graft of claim 14 wherein the matrix has pores wherein the distribution of pore volume as a function of pore size has a mode between 10 micrometers and 25 micrometers.

16. The bone graft of claim 14 wherein the matrix has a porosity between approximately 0.2 and approximately 0.6 .

17. The bone graft of claim 14 wherein the matrix comprises particles of ceramic partially sintered directly to each other.

18. The bone graft of claim 14 wherein the matrix comprises particles of demineralized bone matrix joined to each other by a binder substance.

19. The bone graft of claim 14 wherein the matrix comprises particles of a polymer joined to each other.

20. The bone graft of claim 1 wherein the bone graft comprises a ceramic.

21. The bone graft of claim 1 wherein the bone graft comprises at least one substance selected from the group consisting of hydroxyapatite, tricalcium phosphate and other calcium-phosphorus compounds.

22. The bone graft of claim 1 wherein the bone graft comprises nonresorbable material.

23. The bone graft of claim 1 wherein the bone graft comprises resorbable material.

24. The bone graft of claim 1 wherein the bone graft comprises both nonresorbable and resorbable materials.

25. The bone graft of claim 1 wherein the bone graft comprises both nonresorbable and resorbable materials in different proportions in different places within the bone graft.

26. The bone graft of claim 1, further comprising channels which extend into an interior of the bone graft.

27. The bone graft of claim 1, further comprising channels or patterns on a surface of the bone graft.

28. The bone graft of claim 1 wherein the bone graft comprises a surface having a surface geometry which is different from a geometry at an interior of the bone graft.

29. The bone graft of claim 1 wherein the bone graft comprises a surface having a surface composition which is different from a composition at an interior of the bone graft.

30. The bone graft of claim 1 wherein the bone graft comprises a surface having a surface geometry suitable to face natural bone.

31. The bone graft of claim 1 wherein the bone graft comprises a surface having a surface composition suitable to face natural bone.

32. The bone graft of claim 1, further comprising osteoconductive or osteoinductive substances.

33. The bone graft of claim 1, further comprising substances from a patient's own blood or other biological substances or demineralized bone matrix.

34. The bone graft of claim 1, further comprising a polymer.

35. The bone graft of claim 34 wherein the polymer is a comb polymer.

36. The bone graft of claim 34 wherein the polymer is resorbable.

37. The bone graft of claim 34 wherein the polymer is non-resorbable.

38. The bone graft of claim 1 wherein the bone graft is sterile.

39. The bone graft of claim 1 wherein the bone graft is manufactured at least in part by three dimensional printing.

40. A method of installing a bone graft to augment a sinus, comprising: resecting gingiva; cutting a window in a buccal cortical plate to access the sinus; and installing, in the sinus, a bone graft comprising synthetic material or demineralized bone matrix in a rigid form.

41. The method of claim 40 wherein the bone graft has patient-specific dimensions prior to surgery.

42. The method of claim 40, further comprising, after the cutting of the window but before the installing of the bone graft, removing tissue contained within the sinus.

43. The method of claim 40, further comprising, after the cutting of the window but before the installing of the bone graft, removing a septum of the sinus.

44. The method of claim 40, further comprising, after the cutting of the window but before the installing of the bone graft, cutting within the sinus using a bone-cutting tool which is capable of cutting bone.

45. The method of claim 40, further comprising, after the cutting of the window but before the installing of the bone graft, cutting within the sinus using a bone-cutting tool which is capable of cutting bone and a non-bone-cutting tool which is incapable of cutting bone but capable of cutting soft tissue.

46. The method of claim 40, further comprising, after the cutting of the window but before the installing of the bone graft, applying antiseptic and/or antibiotic.

47. The method of claim 40, further comprising, after the cutting of the window but before the installing of the bone graft, applying a formable filler material between the bone graft and the sinus.

48. The method of claim 40 wherein the bone graft comprises more than one part, all of which parts fitting together to form the bone graft, and wherein the installing of the bone graft comprises bringing in the parts in succession.

49. The method of claim 40, further comprising, after installing the bone graft, attaching the bone graft.

50. The method of claim 40, further comprising, after the installing of the bone graft, installing an implant base through the maxilla and into the bone graft.

51. The method of claim 50 wherein installing the implant base comprises using an implant base template which is unique to a particular patient.

52. The method of claim 51 wherein at least one dimension of the implant base template is determined using radiographic data.

53. The method of claim 40, further comprising after installing the bone graft, applying a surgical membrane.

54. The method of claim 40, further comprising, after all of the other steps, putting the resected gingiva back in place.

55. The method of claim 40 wherein cutting the window is performed using a window template which is unique to a particular patient.

56. The method of claim 55 wherein at least one dimension of the window template is determined using radiographic data.

57. A method of installing a bone graft to augment a sinus, comprising: resecting gingiva; cutting a window in a buccal cortical plate using a patient-unique window template; and installing, in the sinus, a filler.

58. The method of claim 57 wherein at least one dimension of the patient-unique window template is determined using radiographic data.

59. The method of claim 57 wherein the filler is a formable material.

60. The method of claim 57 wherein the filler is a bone graft comprising a rigid material.

61. The method of claim 60 wherein the bone graft comprises synthetic material.

62. The method of claim 60 wherein the bone graft comprises demineralized bone matrix.

63. The method of claim 60 wherein the bone graft has at least one dimension which is selected based on characteristics of a particular site in a particular patient.

64. The method of claim 60 wherein the bone graft has at least one dimension which is selected based on a dimension of an implant base.

65. The method of claim 60 wherein the window template has at least one dimension which is selected based on a dimension of the bone graft.

66. The method of claim 60, further comprising, after the cutting of the window but before the installing of the bone graft, removing tissue contained within the sinus.

67. The method of claim 60, further comprising, after the cutting of the window but before the installing of the bone graft, removing a septum of the sinus.

68. The method of claim 60, further comprising, after the cutting of the window but before the installing of the bone graft, applying antiseptic and/or antibiotic.

69. The method of claim 60, further comprising, after the cutting of the window but before the installing of the bone graft, applying a formable filler material between the bone graft and the sinus.

70. The method of claim 60, further comprising, after installing the bone graft, attaching the bone graft.

71. The method of claim 60, further comprising, after the installing of the bone graft, installing an implant base through the maxilla and into the bone graft.

72. The method of claim 60 wherein installing the implant base comprises using an implant base template which is unique to a particular patient.

73. The method of claim 72 wherein at least one dimension of the implant base template is determined using radiographic data.

74. The method of claim 57, further comprising, after installing the bone graft, applying a surgical membrane.

75. The method of claim 57, further comprising, after all of the other steps, putting the resected gingiva back in place.

76. A method of installing a bone graft to augment a sinus, comprising: resecting gingiva; cutting a window in a buccal cortical plate to access the sinus; and installing, in the sinus, a rigid bone graft; and installing an implant base in the maxilla and the bone graft using an implant base template which is unique to a particular patient.

77. The method of claim 76 wherein at least one dimension of the implant base template is determined using radiographic data.

78. The method of claim 76 wherein the bone graft has at least one dimension which is selected based on characteristics of a particular site in a particular patient.

79. A method of manufacturing a bone graft for augmenting a sinus, comprising spreading successive layers of a powder and three dimensionally printing an article suitable to at least approximately fit the dimensions of a portion of the sinus.

80. The method of claim 79, further comprising, before any other steps, obtaining dimensions from radiographic data.

81. The method of claim 79 wherein the powder comprises demineralized bone matrix.

82. The method of claim 79 wherein the powder comprises ceramic.

83. The method of claim 82, further comprising, after the three dimensional printing, heating the article for a combination of temperature and time sufficient to partially sinter the article.

84. The method of claim 79, further comprising, after all the described steps, introducing an additional substance into pores of the article.

85. The method of claim 84 wherein the additional substance comprises substances from the patient's own blood or other biological substances or demineralized bone matrix.

86. An article manufactured by the method of claim 79.

87. A kit for installing a bone graft in a sinus, comprising: at least one bone graft comprising synthetic material or demineralized bone matrix, and at least one cutting tool.

88. The kit of claim 87 wherein the bone graft has bone graft dimensions which are unique to a particular patient.

89. The kit of claim 87 wherein at least one bone graft dimension is coordinated with dimensions of the sinus.

90. The kit of claim 87, further comprising a patient-unique window template suitable to guide a cutting of a window in a buccal cortical plate.

91. The kit of claim 87, further comprising at least one bone-cutting tool capable of cutting bone and at least one non-bone-cutting tool which is incapable of cutting bone but capable of cutting soft tissue.

92. The kit of claim 87, further comprising a patient-unique implant base template suitable to guide an installing of an implant base.

93. The kit of claim 87, further comprising at least one implant base, and at least one drill for preparing a site for the implant base, and a tool for installing the implant base.

94. The kit of claim 87, further comprising at least one additional article selected from the group consisting of: a carrier for gripping the bone graft, surgical screws, tools for installing surgical screws, formable filler material, antiseptics, antibiotics, a surgical membrane, and sutures.

95. The kit of claim 87 wherein at least some articles in the kit are sterile.
